SEQUENCE LISTING (2 sequences)

SEQ ID NO: 1 - DNA, 20 bp, synthetic construct
  source 1..20: mol_type = other DNA
agagtttgatcatggctcag

SEQ ID NO: 2 - DNA, 18 bp, synthetic construct
  source 1..18: mol_type = other DNA
gtattaccgcggckgctg

(19) United States
(12) Patent Application Publication
(10) Pub. No.: US 2026/0317754 A1
(43) Pub. Date: Oct. 15, 2026

(54) NOVEL EXTRACTION METHOD

(71) Applicant: SVAR LIFE SCIENCE AB, Malmö (SE)

(72) Inventors: Einar MØRK, Lysaker (NO); Anna PRAMHED, BJÄRRED (SE); Krzysztof Sikorski, OSLO (NO); Frida PAULY, SVEDALA (SE)

(21) Appl. No.: 19/485,558

(22) PCT Filed: May 23, 2024
(86) PCT No.: PCT/EP2024/064216

§ 371 Date: Nov. 18, 2025

(30) Foreign Application Priority Data
May 25, 2023 (EP) 23175251.0

(51) Int. Cl. C12N 15/10; C12Q 1/6806

(52) U.S. Cl. CPC C12N 15/1003; C12N 15/1013; C12N 15/1017; C12Q 1/6806

(57) ABSTRACT

Present invention relates to a novel method and use of a buffer for nucleotide extraction from a biological sample.

### FIELD OF INVENTION

Present invention relates to a novel method of extracting polynucleotides such as e.g. DNA or RNA. The invention relates to the use of a buffer for extraction of a polynucleotide, cell lysis and storage of polynucleotide from a biological sample. In a particular aspect, present invention relates to selectively extract DNA from a faecal sample. In a further particular aspect, present invention relates to selectively extract bacterial DNA from a human faecal sample.

### BACKGROUND OF INVENTION

In the art, there are several approaches to extraction and isolation of polynucleotides such as e.g. DNA or RNA. Some of the most common DNA extraction methods include organic extraction, Chelex extraction, and solid phase extraction. These methods consistently yield isolated DNA, but they differ in both the quality and the quantity of DNA yielded. When selecting a DNA extraction method, there are multiple factors to consider, including cost, time, safety, and risk of contamination. Organic extraction involves the addition of and incubation in multiple different chemical solutions; including a lysis step, a phenol chloroform extraction, an ethanol precipitation, and washing steps. Organic extraction is often used in laboratories because it is cheap, and it yields large quantities of pure DNA. Though it is easy, there are many steps involved, and it takes longer than other methods. It also involves the unfavourable use of the toxic chemicals phenol and chloroform, and there is an increased risk of contamination due to transferring the DNA between multiple tubes. Several protocols based on organic extraction of DNA were effectively developed decades ago, though improved and more practical versions of these protocols have also been developed and published in the last years.

The Chelex® extraction method involves adding the Chelex® resin to the sample, boiling the solution, then vortexing and centrifuging it. The cellular materials bind to the Chelex® beads, while the DNA is available in the supernatant. The Chelex® method is much faster and simpler than organic extraction, and it only requires one tube, which decreases the risk of DNA contamination. Unfortunately, Chelex® extraction does not yield as much quantity and the DNA yielded is single-stranded, which means it can only be used for PCR-based analyses and not for RFLP.

Solid phase extraction such as using a spin-column based extraction method takes advantage of the fact that DNA binds to silica. The sample containing DNA is added to a column containing a silica gel or silica beads and chaotropic salts. The chaotropic salts disrupt the hydrogen bonding between strands and facilitate binding of the DNA to silica by causing the nucleic acids to become hydrophobic. This exposes the phosphate residues so they are available for adsorption. The DNA binds to the silica, while the rest of the solution is washed out using ethanol to remove chaotropic salts and other unnecessary constituents. The DNA can then be rehydrated with aqueous low salt solutions allowing for elution of the DNA from the beads.

This method yields high-quality, largely double-stranded DNA which can be used for both PCR and RFLP analysis. This procedure can be automated and has a high throughput, although lower than the phenol-chloroform method. This is a one-step method i.e the entire procedure is completed in one tube. This lowers the risk of contamination making it very useful for forensic extraction of DNA. Multiple solid phase extraction commercial kits are manufactured and marketed by different companies; the only problem is that they are more expensive than organic extraction or Chelex® extraction.

### SUMMARY OF THE INVENTION

Present invention provides a simple and easy method for extracting polynucleotide material from a biological sample, keeping the polynucleotide material intact prior to analysis, and overcoming the above-mentioned disadvantages.

Present invention relates to the use of an extraction and storage buffer in the methods described herein.

The buffer may comprise e.g. an aqueous extraction medium further comprising at least one of:
i) one or more dissociating agents,
ii) one or more disaggregating agents,
iii) and/or one or more chelating agents.

The method according to the invention may comprise the steps of:
a) providing a biological sample,
b) contacting said biological sample with an amount of the extraction buffer according to the invention,
c) removal of any solids from the sample from step b),
d) PCR analysis of the sample from step c) isolating DNA/RNA with e.g. magnetic beads, silica spin column or other relevant methods for polynucleotide isolation, and perform the PCR analysis on this material with relevant probes

Present invention also relates to a method for determining or elucidation of a microbiome. Consequently, present invention relates to detection of a community of microorganisms (such as e.g. bacteria, fungi, and viruses) that inhabit a particular environment and especially the collection of microorganisms living in or on the human body (or animal body) or any part or compartment of the human body (or animal body).

Thus, present invention relates to use of a buffer composition for isolation or determination of a microbiome.

The buffer composition for use in i.a. determination of a microbiome may comprise at least one of:
i) one or more dissociating agents,
ii) one or more disaggregating agents,
iii) and/or one or more chelating agents.

### FIGURES

FIG. 1 illustrates the score of 10 samples analyzed in the GA-map™ Dysbiosis test using two different sampling devices. Scores above 2 indicates a microbiota profile that differs from the reference population and score 3 indicates a mild dysbiosis.

FIG. 2 illustrates electrophoretic analysis of PCR products obtained with primers specific for the bacterial 16S rRNA gene. Briefly, a human fecal sample was processed either using EasyExtract (EE) using the buffer according to the invention, Qiagen PowerFecal Pro DNA kit (DF) or EasyExtract followed by Qiagen DNEasy PowerClean Pro clean-up kit (DP). The samples were then amplified in a PCR reaction neat, or diluted as indicated. A comparable product is present in all samples indicating the possibility to use the EasyExtract sample directly in PCR to identify bacterial species by 16S rRNA PCR.

FIG. 3 illustrates electrophoretic analysis of PCR products obtained with primers specific for the human GAPDH gene. Briefly, a human fecal sample was processed either using EasyExtract (EE) using the buffer according to the invention, Qiagen PowerFecal Pro DNA kit (DF) or EasyExtract followed by Qiagen DNEasy PowerClean Pro clean-up kit (DP). The samples were then amplified in a PCR reaction neat, or diluted as indicated. There is no visible product present in the EE sample, indicating that no detectable human DNA is present.

### DETAILED DESCRIPTION OF THE INVENTION

As disclosed herein, present invention relates to a buffer composition.

The buffer composition according to the invention for use in isolation or determination of a microbiome may comprise at least one of:
i) one or more dissociating agents,
ii) one or more disaggregating agents,
iii) and/or one or more chelating agents.

In another aspect, present invention relates to use of a buffer composition for extraction and storage of a polynucleotide. The polynucleotide may be a DNA or an RNA or a fragment thereof.

The buffer composition according to the invention for use in extraction of a polynucleotide may comprise at least one of:
i) one or more dissociating agents,
ii) one or more disaggregating agents,
iii) and/or one or more chelating agents.

As is apparent herein, the buffer composition may be used for extracting one or more polynucleotides from a biological sample. The sample may in principle be any type of biological sample from any type of organ or tissue.

In a particular aspect, the biological sample may be a faecal sample.

In a further particular aspect, the faecal sample may be a human faecal sample.

In the various aspects of the invention, the one or more dissociating agents may be an enzyme compound or a non-enzymatic compound. In another aspect, the dissociating agent may be a detergent or a detergent like compound.

In one aspect, present invention relates to use of the buffer composition for selectively extracting DNA and/or RNA originating from microorganisms, i.e. not human DNA in a biological sample.

In one aspect, the DNA and/or RNA may be of bacterial or viral, or fungal origin or any combination thereof.

In one aspect, the DNA and/or RNA may be of bacterial, viral, or fungal origin or any combination thereof.

In one aspect, the DNA and/or RNA may be of bacterial origin.

In one aspect, the DNA and/or RNA may be of viral origin.

In one aspect, the DNA and/or RNA may be of fungal origin.

In one aspect, the detergent or detergent like compound may be nonionic, anionic, cationic, or zwitterionic, based on their hydrophilic head group feature. Non-limiting examples of detergent are e.g. nonionic detergents like Triton X-100 and zwitterionic detergents like CHAPS (3-[(3-cholamidopropyl)dimethylammonio]-1-propanesulfonate) which are nondenaturing (will not disrupt protein functions). Ionic detergents like sodium dodecyl sulfate (SDS) and cationic detergents like ethyl trimethyl ammonium bromide are denaturing (will disrupt protein functions). Other examples are CHAPSO (3-([3-Cholamidopropyl]dimethylammonio)-2-hydroxy-1-propanesulfonate), octyl glucoside, octyl thioglucoside, Tween-20 (polyoxyethylenesorbitan monolaurate), Tween-80 (Polyoxyethylenesorbitan monooleate), NP-40 (nonyl phenoxypolyethoxylethanol), Triton X-114, Brij-35 (polyoxyethyleneglycol dodecyl ether), Brij-58 (polyethylene glycol hexadecyl ether), ETMAB (ethyl trimethylammonium bromide), lauryl trimethyl ammonium bromide (LTAB), lauryl trimethylammonium chloride (LTAC) or the likes.

In one aspect, the dissociating agent may be urea or sodium dodecyl sulphate (SDS) or any compound comprising urea and/or SDS.

In several aspects of the invention, the one or more disaggregating agents may be e.g. include neutral protease, collagenase, trypsin, lipase, hyaluronidase, deoxyribonuclease, BSA (Bovine Serum Albumin) and pepsin, and among them, collagenase is preferable.

In another preferred aspect, the disaggregating agent may be BSA (Bovine Serum Albumin).

In several aspects of the invention, the buffer composition may comprise one or more chelators. Chelating agents are chemical compounds whose structures permit the attachment of their two or more donor atoms (or sites) to the same metal ion simultaneously and produce one or more rings. Any compound capable of forming a chelate may be envisaged in the buffer composition according to the invention. In several aspects of the invention, the one or more chelating agents may be e.g. a citrate, a phosphate, EDTA (ethylenediaminetetraacetic acid), EGTA (ethylene glycol-bis(β-aminoethyl ether)-N,N,N′,N′-tetraacetic acid), DTPA (penta (carboxymethyl) diethylenetriamine), HEDTA (N-(2-hydroxyethyl)ethylenediamine-N,N′, N′-triacetic acid), or NTA (N,N-bis(carboxymethyl)glycine) or any salts thereof.

In one aspect, the chelator is a citrate. In another aspect, the chelator is a phosphate, such as e.g. sodium phosphate or potassium phosphate.

The pH of the buffer composition according to the invention may be in any range of e.g. about 5.5 to about 11.5. In one aspect, the pH may be in any range of about 5 to about 10.

In yet a further aspect, the pH may be about 8. In another aspect, the pH may be about 7.8 to about 8.2.

In aspect, the pH of the solution in which the biological sample is extracted may be about 7.8 to about 8.2, or about 8.0

In one aspect, the buffer composition according to the invention may further comprise BSA, Tris, HEPES or the likes. In one particular aspect, the buffer may be e.g. Tris.

In one aspect, the buffer composition according to the invention may further comprise a salt such as e.g. NaCl, KCl, and (NH₄)₂SO₄. Another non-limiting example may be e.g. CaCl₂).

In a further aspect, the buffer composition may comprise a reducing agent such as e.g. an azide (such as e.g. sodium azide, NaN₃) or DDT (dithiothreitol), β-mercaptoethanol, TCEP (tris(2-carboxyethyl)phosphine), or the likes.

In one particular aspect, the reducing agent may be e.g. NaN₃.

In another aspect, the buffer composition may comprise one or more preservatives such as e.g. methylchloroisothiazolinone or methylisothiazolinone or a combination thereof. The combination of said thiazolones is marketed under tradename Kathon CG. Specifically, Kathon CG comprises a combination of 5-chloro-2-methyl-4-isothiazolin-3-one (CAS #26172-55-4) and 2-methyl-4-isothiazolin-3-one (CAS #2682-20-4) in amounts of 1.15% and 0.35% respectively.

As mentioned herein, the buffer may be an aqueous buffer and consequently employing water as the major solvent. However, the buffer may also comprise further suitable solvents or co-solvents.

In one aspect, the buffer composition according to the invention may comprise:
i) one or more dissociating agents,
ii) one or more disaggregating agents,
iii) and/or one or more chelating agents, and
iv) one or more preservatives, or reducing agents, or antimicrobial agents, or a combination thereof.

Thus, in one aspect, the buffer according to the invention may comprise at least one of:
i) one or more dissociating agents, selected from urea or sodium dodecyl sulphate (SDS),
ii) one or more disaggregating agents, wherein the disaggregating agent is BSA,
iii) and/or one or more chelating agents, selected from citrate or phosphate compound.

In a further aspect, the buffer according to the invention may comprise at least one of:
i) one or more dissociating agents, selected from urea or sodium dodecyl sulphate (SDS),
ii) one or more disaggregating agents, wherein the disaggregating agent is BSA,
iii) and/or one or more chelating agents, selected from citrate or phosphate compound,
iv) one or more preservatives, or reducing agents or antimicrobial agents elected from an azide salt and/or one or more thiazolones selected from e.g. methylchloroisothiazolinone or methylisothiazolinone or a combination thereof.

In yet a further specific aspect, the buffer according to the invention may comprise or consist of at least one of:
Sodium Citrate,
Tris: NaOH,
Urea,
BSA,
CaCl₂),
NaN₃,
Kathon (methylchloroisothiazolinone and methylisothiazolinone).

As mentioned herein, present invention relates to use of a buffer composition in a method which may comprise extraction of a polynucleotide from a biological sample. The biological sample may in principle be of any nature or tissue origin, such as e.g. solid tissue, blood sample or sera. In a preferred aspect, the sample is a faecal sample.

Present invention also relates to a method for extracting a polynucleotide from a biological sample. The biological sample may in principle be of any nature or tissue origin such as e.g. solid tissue, blood sample or sera. In a preferred aspect, the sample is a faecal sample.

In one aspect, the buffer and/or method according to the invention enables selective extraction of one or more polynucleotide. One specific example may be e.g. that a biological sample is taken from an organism or subject for extraction or isolation of one or more polynucleotides belonging to a different species. Thus, the organism or subject may be regarded as a host for one or more other/different species, wherein the buffer and method according to the invention enables specific isolation or extraction of non-host polynucleotides/DNA. One non-limiting example, is e.g. a faecal sample taken from a human wherein bacterial DNA is selectively extracted. In the context of the invention, this means that e.g. bacterial (or viral, or fungal) DNA may be selectively extracted from a faecal sample with low levels of human DNA or levels of human DNA below the limit of detection according to prevailing art.

Moreover, by the use of the extraction buffer according to the invention, a more facile procedure is provided. Specifically, by the use of the buffer according to the invention, a lesser degree of dilution is required. Normally, several orders of dilution is required. However, by the aid of presently claimed invention, a lesser degree of dilution may be employed and still offer a reliable result without interference of other factors.

In a further aspect, it has also been found that extracted samples may be stored for longer periods of time without degrading the extracted DNA. This also result in a more efficient handling and not requiring immediate analysis of extracted samples.

According to the invention, the method may comprise one or more steps of:
a) providing a biological sample,
b) contacting said biological sample with an amount of the buffer according to the invention,
c) removal of any solids from the sample from step b).

The method may further comprise
d) PCR analysis of the sample from step c).

In yet a further aspect, the method may comprise isolating DNA/RNA with e.g. magnetic beads, silica spin column or other relevant methods for polynucleotide isolation, and perform the PCR analysis on this material with relevant probes.

According to the invention, the biological sample may be mixed in any suitable manner with the buffer composition according to the invention. In a further aspect, the biological sample may be subjected to a homogenisation step either prior to being mixed with the buffer composition or may be subjected to homogenisation together with the buffer composition according to the invention. Thus contacting the biological sample with the buffer composition may be effected by simple mixing, or homogenisation etc.

The biological sample may be provided in a solid form, or as a liquid or may have been previously frozen and subsequently thawed prior to being mixed with the buffer composition.

Once the biological sample has been contacted with the buffer composition, optionally, the mixture may be left standing for a certain period of time ranging from about 1 minute to about 24 h. Alternatively, the mixture may be directly processed to the next process step.

Removal of solids form the mixture obtained from step b) may be effected by any suitable method known in the art, such as e.g. filtration or centrifugation or even sedimentation as deemed appropriate.

The resulting liquid from step c) may be further processed and may comprise analysis of the obtained polynucleotide. Such analysis may comprise any type of characterisation of the polynucleotide, such as e.g. determination of the nucleotide sequence. Such methods may include PCR analysis.

As mentioned herein, the method may comprise isolating the polynucleotide resulting from the extraction. Consequently, isolation may take place by any known method in the art such as e.g. magnetic beads, silica spin column or other relevant methods for polynucleotide isolation.

As is apparent to a person skilled in the art, the method according to the invention may further comprise one or more of PCR quantification control, clean-up of amplified DNA, end-labeling of probe set, hybridization, and signal detection.

In specific embodiments, present invention also relates to the following items;
1. Use of a buffer composition for extraction of one or more polynucleotides from a biological sample, the composition comprising;
i) one or more dissociating agents,
ii) one or more disaggregating agents,
iii) and/or one or more chelating agents.
2. Use according to item 1, wherein the one or more dissociating agents is a detergent or a detergent like compound, or selected from nonionic, anionic, cationic, or zwitterionic, based on their hydrophilic head group feature. Non-limiting examples of detergent are e.g. nonionic detergents like Triton X-100 and zwitterionic detergents like CHAPS (3-[(3-cholamidopropyl)dimethylammonio]-1-propanesulfonate) which are nondenaturing (will not disrupt protein functions), sodium dodecyl sulfate (SDS) and cationic detergents like ethyl trimethyl ammonium bromide are denaturing (will disrupt protein functions), CHAPSO (3-([3-Cholamidopropyl]dimethylammonio)-2-hydroxy-1-propanesulfonate), octyl glucoside, octyl thioglucoside, Tween-20 (polyoxyethylenesorbitan monolaurate), Tween-80 (Polyoxyethylenesorbitan monooleate), NP-40 (nonyl phenoxypolyethoxylethanol), Triton X-114, Brij-35 (polyoxyethyleneglycol dodecyl ether), Brij-58 (polyethylene glycol hexadecyl ether), ETMAB (ethyl trimethylammonium bromide), lauryl trimethyl ammonium bromide (LTAB), lauryl trimethylammonium chloride (LTAC).
3. Use according to any of the preceding items, wherein the one or more disaggregating agents are selected from neutral protease, collagenase, trypsin, lipase, hyaluronidase, deoxyribonuclease, BSA (Bovine Serum Albumin) and pepsin, or a collagenase.
4. Use according to any of the preceding items, wherein the one or more chelating agents are selected from a citrate, a phosphate, EDTA (ethylenediaminetetraacetic acid), EGTA (ethylene glycol-bis(β-aminoethyl ether)-N,N,N′,N′-tetraacetic acid), DTPA (penta (carboxymethyl) diethylenetriamine), HEDTA (N-(2-hydroxyethyl)ethylenediamine-N, N′, N′-triacetic acid), or NTA (N,N-bis(carboxymethyl)glycine) or any salts thereof.
5. Use according to any of the preceding items, wherein the composition has a pH in any range of e.g. about 5.5 to about 11.5. In one aspect, the pH may be in any range of about 5 to about 10, or the pH may be about 8, or the pH may be about 7.8 to about 8.2.
6. Use according to any of the preceding items, wherein the dissociating agent is urea or sodium dodecyl sulphate (SDS), and wherein the disaggregating agent is BSA (Bovine Serum Albumin), and wherein the chelating agent is a citrate or a phosphate.
7. Use according to any of the preceding items, wherein the composition further comprises one or more of; Tris, HEPES, a salt such as e.g. NaCl, KCl, and (NH₄)₂SO₄, a reducing agent such as e.g. an azide (such as e.g. sodium azide, NaN₃) or DDT (dithiothreitol), β-mercaptoethanol, orTCEP (tris(2-carboxyethyl)phosphine),
8. Use according to any of the preceding items, wherein the biological sample is of any nature or tissue origin, such as e.g. solid tissue, blood sample or sera, or the sample is a faecal sample.
9. A method of extracting one or more polynucleotides from a biological sample, the method comprising the steps of;
a) providing a biological sample,
b) contacting said biological sample with an amount of the buffer composition as defined in any of the preceding claims,
c) removal of any solids from the sample from step b), to thereby acquire a sample into which said one or more polynucleotides are extracted.
10. The method according to item 9, wherein the method further comprises any analysis of the obtained one or more polynucleotides comprising any type of characterisation of the polynucleotide, such as e.g. determination of the nucleotide sequence including PCR analysis.
11. The method according to any one of items 9-10, wherein the method further comprises isolating the one or more obtained polynucleotides and further comprising methods with the aid of magnetic beads, silica spin column or other relevant methods for polynucleotide isolation.
12. The method according to any one of items 9-11, wherein the method further comprises one or more of PCR quantification control, clean-up of amplified DNA, end-labeling of probe set, hybridization, and signal detection.

### Experimental Section

The invention is further illustrates in the following non-limiting examples.

### Example 1

### Materials and Methods

Our study is based on 10 volunteers taking stool samples with both a device according to present invention and GA's sampling devices as a comparison. The inventors of present invention have designed a device comprising a dip-stick where grooves are filled with stool before inserting the stick into a tube containing sample buffer which is the buffer according to the invention. The device collects approximately 30 μl sample. Another device (comparative device) was used for comparison and has a spoon shaped part and collects considerably more material, approximately 10 mL stool (2 teaspoons) transferred into an empty sample tube.

Samples were collected by the participants according to the supplier's instructions and kept refrigerated until the next day when all samples were collected and frozen. Samples were shipped frozen for genetic analysis (denoted as GA).

The summarized result is reported as a dysbiosis index on a scale from 1 to 5 where an index above 2 indicates a microbiota profile that differs from the reference population and score 3 indicates a mild dysbiosis.

An algorithm is used to calculate the dysbiosis index based on the abundance of the analyzed bacterial strains, where “normal” level corresponds to the signal level of approximately 80% of the normal healthy population. A scoring system is used where 0 is normal, and high or low abundance of the individual strains are scored between-3 and +3.

The buffer composition used in the examples is as follows:
0.1 M NaCitrate
0.1 M Tris: NaOH pH 8.0
1M Urea
0.5% BSA
10 mM CaCl2
0.09% NaN₃
0.1% Kathon (methylchloroisothiazolinone and methylisothiazolinone).

### Results

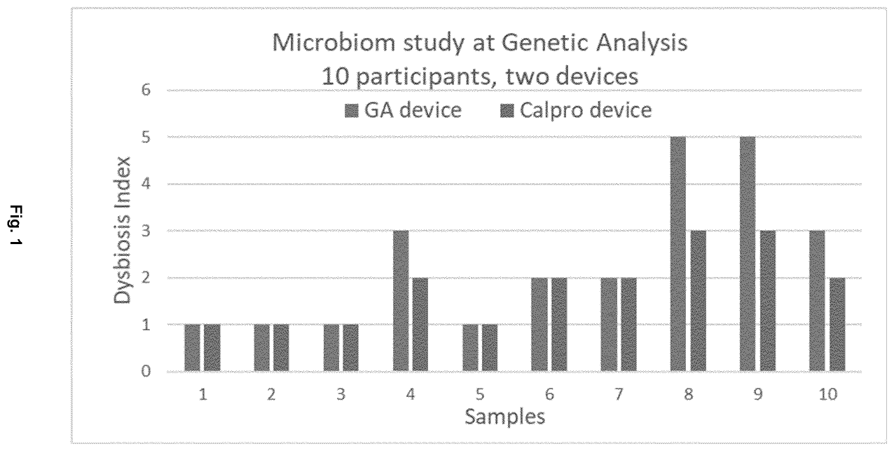

In comparison between the two sampling devices, 6 out of 10 samples resulted in an identical dysbiosis index on the 1 to 5 scale. The 4 samples that gave differing results all scored lower with the device designed by the inventors of present invention (buffer according to the invention) than with the device used for comparison (FIG. 1).

The dysbiosis index is based on the abundance scoring of the individual bacterial strains. Out of the 390 individual scores (39 bacterial strains×10 samples), 307 (79%) gave the same result between the two devices while 83 (21%) deviated. The deviating results all scored lower with the device designed by the inventors of present invention, than with comparative device (Table 1).

Signal data for individual readings were generally lower using the device designed by the inventors of present invention compare to the comparative devise partly explaining the lower scores for some of the samples when using the device designed by the inventors of present invention. Signal data for one sample is presented in table 2.

TABLE 1
Illustrates the abundance of individual bacterial strains is
reported in a scoring system (ranging from −3 to +3*)
where 0 is within normal reference range. The majority of strains
scored identical between the two devices (PI; Present Invention,
and GA respectively), exemplified with results from sample 7
and sample 9 below. Deviations marked in bold faced font.

| | Sample | Sample | Sample | Sample |
|---|---|---|---|---|
| | 7, GA | 7, PI | 9, GA | 9, PI |
| Actinobacteria | −2 | −2 | 0 | −1 |
| Actinomycetales | 0 | 0 | 0 | 0 |
| Bifidobacterium spp. | −1 | −1 | 0 | −1 |
| Alistipes | 0 | 0 | 0 | 0 |
| Alistipes onderdonkii | −2 | −2 | 0 | 0 |
| Bacteroides fragilis | 0 | 0 | 0 | 0 |
| Bacteroides spp. & Prevotella spp. | 0 | 0 | 0 | 0 |
| Bacteroides stercoris | 0 | 0 | 1 | 1 |
| Bacteroides zoogleoformans | 0 | 0 | 0 | 0 |
| Parabacteroides johnsonii | 0 | 0 | 0 | 0 |
| Parabacteroides spp. | 0 | −1 | 0 | 0 |
| Firmicutes | 0 | 0 | 0 | 0 |
| Bacilli | 0 | −1 | −1 | −1 |
| Catenibacterium mitsuokai | 0 | 0 | 0 | 0 |
| Clostridia | 0 | 0 | 0 | 0 |
| Clostridium sp. | 0 | 0 | 0 | 0 |
| Dialister invisus | 0 | 0 | 0 | 0 |
| Dialister invisus & Megasphaera | 0 | 0 | 0 | 0 |
| micronuciformis | | | | |
| Dorea spp. | 0 | 0 | 0 | 0 |
| Eubacterium biforme | 0 | 0 | 0 | 0 |
| Eubacterium hallii | 0 | −1 | 0 | −1 |
| Eubacterium rectale | 0 | 0 | 1 | 0 |
| Eubacterium siraeum | −2 | −2 | −2 | −2 |
| Faecalibacterium prausnitzii | −1 | −1 | 0 | 0 |
| Lachnospiraceae | 1 | 0 | 1 | 0 |
| Lactobacillus ruminis & | 0 | 0 | 0 | 0 |
| Pediococcus acidilactici | | | | |
| Lactobacillus spp. | 0 | 0 | 0 | 0 |
| Phascolarctobacterium sp. | 1 | 0 | 0 | 0 |
| Ruminococcus albus & R. bromii | 0 | 0 | 0 | 0 |
| Ruminococcus gnavus | 0 | 0 | 3 | 2 |
| Streptococcus agalactiae & | 0 | 0 | 1 | 0 |
| Eubacterium rectale | | | | |
| Streptococcus salivarius ssp. | 0 | 0 | 0 | 0 |
| thermophilus & S. sanguinis | | | | |
| Streptococcus salivarius ssp. | −1 | −1 | −1 | −1 |
| thermophilus | | | | |
| Streptococcus spp. | 0 | 0 | 0 | 0 |
| Veillonella spp. | 0 | −1 | 0 | −1 |
| Proteobacteria | 0 | 0 | 3 | 1 |
| Shigella spp. & Escherichia spp. | −1 | −1 | 3 | 1 |
| Mycoplasma hominis | −1 | −1 | −1 | −1 |
| Akkermansia muciniphila | 1 | 0 | 0 | 0 |

*A scoring system is used where 0 is normal, and high or low abundance of the individual strains are scored between −3 and +3.

TABLE 2
Illustrates the raw data signals were generally lower
with the device designed by the inventors of present
invention compared to the comparative device, exemplified
with results from sample 7 and sample 9 below.

| | Sample 9, | Sample |
|---|---|---|
| | GA | 9, PI |
| Actinobacteria | 206 | 66 |
| Actinomycetales | 10 | 12 |
| Bifidobacterium spp. | 108 | 29 |
| Alistipes | 176 | 156 |
| Alistipes onderdonkii | 39 | 28 |
| Bacteroides fragilis | 6 | 6 |
| Bacteroides pectinophilus | −1 | 12 |
| Bacteroides spp. | 39 | 18 |
| Bacteroides spp. & Prevotella spp. | 773 | 787 |
| Bacteroides stercoris | 24 | 31 |
| Bacteroides zoogleoformans | 27 | 17 |
| Parabacteroides johnsonii | 22 | 15 |
| Parabacteroides spp. | 91 | 64 |
| Firmicutes | 485 | 644 |
| Bacilli | 55 | 36 |
| Catenibacterium mitsuokai | −1 | −1 |
| Clostridia | 585 | 562 |
| Clostridium methylpentosum | 20 | 9 |
| Clostridium sp. | −1 | −1 |
| Coprobacillus cateniformis | 13 | 17 |
| Dialister invisus | 14 | 18 |
| Dialister invisus & Megasphaera | 6 | 5 |
| micronuciformis | | |
| Dorea spp. | 44 | 21 |
| Eubacterium biforme | −1 | −1 |
| Eubacterium hallii | 126 | 20 |
| Eubacterium rectale | 334 | 213 |
| Eubacterium siraeum | 16 | 22 |
| Faecalibacterium prausnitzii | 392 | 352 |
| Lachnospiraceae | 1131 | 918 |
| Lactobacillus ruminis & | −1 | −1 |
| Pediococcus acidilactici | | |
| Lactobacillus spp. | 12 | 8 |
| Lactobacillus spp. 2 | −1 | 8 |
| Phascolarctobacterium sp. | 8 | −1 |
| Ruminococcus albus & R. bromii | 197 | 42 |
| Ruminococcus gnavus | 497 | 320 |
| Streptococcus agalactiae and | 170 | 97 |
| Eubacterium rectale | | |
| Streptococcus salivarius ssp. | 9 | 10 |
| thermophilus and S. sanguinis | | |
| Streptococcus salivarius | 11 | 9 |
| ssp. thermophilus | | |
| Streptococcus spp. | 5 | 6 |
| Streptococcus spp. 2 | 10 | 10 |

### Example 2

The same buffer compositions as described in Example 1 above was employed.

### Background and Experiment Objective

Bacterial 16S rRNA gene sequence exhibits large variation allowing for identification of bacterial species and strains. At the same time analysis of microbiome composition in the human gut has emerged as a promising new field, both for diagnostic and therapeutic purposes. The extraction device according to the invention allows for simple preparation of extracts from fecal samples in clinical laboratories and at patients' home. The device has been designed and validated for use with immunochemical assays for measurement of calprotectin.

This experiment was designed to investigate if the device is suitable for analysis of microbiome composition together with the buffer according to the invention.

### Methods

Extracts were prepared from a fecal sample obtained from a healthy donor:
One extract was prepared using the extraction device, according to the manufacturer's instruction. Briefly, grooves of the sampling stick were filled with fecal matter and the stick was replaced in the device comprising a container filled with the buffer solution. The device was then vortexed for 3 minutes. The devices were then centrifuged for 10 min at 1000 rpm to remove any particulate matter and the extract was frozen at −20° C. for later analysis.
Second extract was prepared using the Qiagen PowerFecal Pro DNA kit according to the manufacturer's instruction and the resulting DNA sample was kept at −20° C. until analysis.
The third sample was prepared by processing the extract of the deviceextract, prepared as above, using the Qiagen DNeasy PowerClean Pro Clean-up kit according to the manufacturer's instruction. The resulting DNA sample was kept at −20° C. until analysis.
PCR amplification of the 16s rRNA region:
Primers used: forward AGAGTTTGATCATGGCTCAG and reverse GTATTACCGCGGCKGCTG
Control reactions: T1-colony picked from an agar plate and mixed in pure water, T2-DNA extract from a human HEK cell line
PCR conditions: hybridization temperature 62° C., extension time 15 sec, 30 cycles. PCR buffer: Buffer GC rich (ThermoFisher), 3% DMSO (ThermoFisher), Phusion DNA polymerase (ThermoFisher)

### Conclusions and Results

Based on the above results we conclude it is possible to conduct amplification of the bacterial 16S rRNA gene directly from a sample obtained through the device comprising the buffer composition. Undiluted sample contains PCR inhibitors resulting in complete inhibition of the amplification reaction but already a 10-fold dilution of the sample results in good amplification. At the same time, there is no detectable human DNA in the sample, at least by standard PCR. There is a very weak product present in the DP-Pur lane, indicating that a qPCR technique might be able to detect human DNA also in the unpurified sample using the buffer solution.

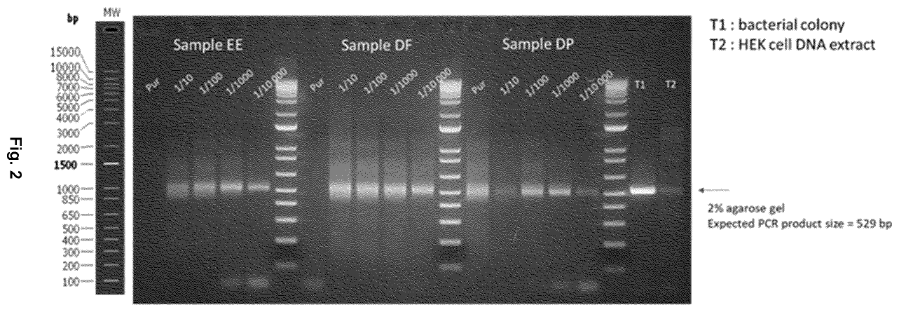

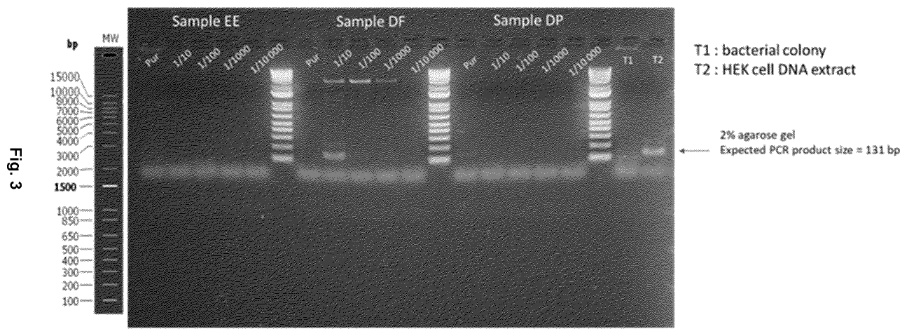

The results of Example 2 are illustrated in FIG. 2 and FIG. 3 respectively. The Example clearly illustrates the selective extraction of bacterial DNA and that a low dilution of the sample may be employed in order to obtain a reliable result.

1. Use of a buffer composition for extraction of one or more polynucleotides from a biological sample, the composition comprising;
i) one or more dissociating agents, selected from urea or sodium dodecyl sulphate (SDS),
ii) one or more disaggregating agents wherein the disaggregating agent is Bovine Serum Albumin (BSA),
iii) and/or one or more chelating agents, selected from a citrate or phosphate compound, and further comprising
iv) one or more preservatives, or reducing agents or antimicrobial agents selected from one or more azide salt and/or one or more thiazolones.

2. Use according to claim 1, wherein the buffer comprise or consist of at least one of:
Sodium Citrate,
Tris: NaOH,
Urea,
BSA,
CaCl₂),
NaN₃,
Kathon (methylchloroisothiazolinone and methylisothiazolinone).

3. (canceled)

4. (canceled)

5. (canceled)

6. (canceled)

7. (canceled)

8. (canceled)

9. Use according to claim 1, wherein the composition has a pH in any range of e.g. about 5.5 to about 11.5. In one aspect, the pH may be in any range of about 5 to about 10, or the pH may be about 8, or the pH may be about 7.8 to about 8.2.

10. (canceled)

11. (canceled)

12. (canceled)

13. (canceled)

14. (canceled)

15. Use according to claim 1, wherein the biological sample is of any nature or tissue origin, such as e.g. solid tissue, blood sample or sera, or the sample is a faecal sample.

16. Use according to claim 1, wherein the biological sample is a faecal sample, such as e.g. a human faecal sample.

17. (canceled)

18. (canceled)

19. A method of extracting one or more polynucleotides from a biological sample, the method comprising the steps of;
a) providing a biological sample,
b) contacting said biological sample with an amount of the buffer composition as defined in any of the preceding claims,
c) removal of any solids from the sample from step b),
to thereby acquire a sample into which said one or more polynucleotides are extracted.

20. The method according to claim 19, wherein the method further comprises any analysis of the obtained one or more polynucleotides comprising any type of characterisation of the polynucleotide, such as e.g. determination of the nucleotide sequence including PCR analysis.

21. The method according to claim 19, wherein the method further comprises isolating the one or more obtained polynucleotides and further comprising methods with the aid of magnetic beads, silica spin column or other relevant methods for polynucleotide isolation.

22. The method according to claim 19, wherein the method further comprises one or more of PCR quantification control, clean-up of amplified DNA, end-labeling of probe set, hybridization, and signal detection.
